(19) United States
(12) Patent Application Publication
(10) Pub. No.: US 2017/0374498 A1
(43) Pub. Date: Dec. 28, 2017

(54) GENERIC SOFTWARE-BASED PERCEPTION RECORDER, VISUALIZER, AND EMOTIONS DATA ANALYZER

(71) Applicants: Shani Markus, Mountain View, CA (US); Manjula Dissanayake, Netherby (AU); Andun Sameera LiyanaGunawardena, Weligama (SR); Sachintha Rajitha Ponnamperuma, Hasitha (SR)

(72) Inventors: Shani Markus, Mountain View, CA (US); Manjula Dissanayake, Netherby (AU); Andun Sameera LiyanaGunawardena, Weligama (SR); Sachintha Rajitha Ponnamperuma, Hasitha (SR)

(21) Appl. No.: 15/141,833

(22) Filed: Apr. 29, 2016

(51) Int. Cl. H04W 4/02; G06Q 50/00; H04L 12/58; A61B 5/16

(52) U.S. Cl. CPC H04W 4/02; H04L 51/32; G06Q 50/01; A61B 5/165

(57) ABSTRACT

The present disclosure relates to a sophisticated system and method of transmitting individual feelings, emotions, and perceptions. The system and method consistent with the present disclosure includes capturing emotions, feelings, or perceptions in a contextual context. The emotions, feelings, or perceptions may be dispatched from a user using an electronic communications device such as a smartphone.

### CROSS-REFERENCE TO RELATED APPLICATION

This application claims the benefit of U.S. Provisional Application No. 62/126,452 filed Feb. 28, 2015 which may be incorporated herein by reference.

### FIELD OF THE DISCLOSURE

The present disclosure relates to a sophisticated system and method of transmitting individual feelings, emotions, and perceptions.

### BRIEF DESCRIPTION OF THE DRAWINGS

To facilitate understanding, identical reference numerals have been used, wherever possible, to designate identical elements that are common to the figures. The drawings are not to scale and the relative dimensions of various elements in the drawings are depicted schematically and not necessarily to scale. The techniques of the present disclosure may readily be understood by considering the following detailed description in conjunction with the accompanying drawings, in which:

FIG. 1 is an illustration of a solution platform for a system consistent with the present disclosure;

FIG. 2 is a schematic layout which illustrates an overview of a solution platform's server-side process;

FIG. 3 is a schematic layout which illustrates a solution platform's client-side process;

FIG. 4 is a flowchart of a method of creating and publishing emotives;

FIG. 5 is a device which displays an interface for selecting and publishing emotives;

FIG. 6 is a depiction of a dashboard which displays emotive analytics and related data;

FIG. 7 is an illustration of a first use case of a perception tracker at a live event;

FIG. 8 is an illustration of a second use case of a perception tracker implemented for a TV show;

FIG. 9 is an illustration of a third use case of a perception tracker implemented for a kiosk; and

FIG. 10 is a depiction of a perception tracker embedded in a web page.

### DETAILED DESCRIPTION

Before the present disclosure is described in detail, it is to be understood that, unless otherwise indicated, this disclosure is not limited to specific procedures or articles, whether described or not.

It is further to be understood that the terminology used herein is for the purpose of describing particular embodiments only and is not intended to limit the scope of the present disclosure.

It must be noted that as used herein and in the claims, the singular forms “a,” and “the” include plural referents unless the context clearly dictates otherwise. Thus, for example, reference to “an enclosure” also includes two or more enclosures, and so forth.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure. The term “about” generally refers to ±10% of a stated value.

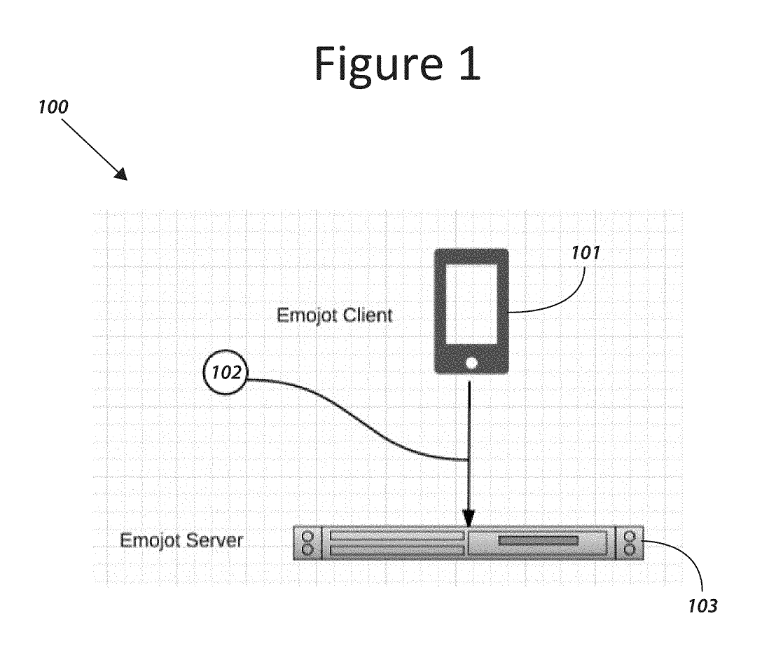

FIG. 1 is an illustration of a solution platform 100 for a system consistent with the present disclosure. Solution platform 100 includes an Emojot 101 client such as a smartphone or other electronic communications device 101. Utilizing the Emojot client 101 allows a user (e.g., subscribing users0 to transmit an emotive to effect emoting 102), which represents the user's feelings or perceptions, to a server-side computational and storage device (e.g., Emojot server 103) to enable crowd-sourced perception visualization and in-depth perception analysis. In some embodiments of the present disclosure, emotives are icons which represent an emotion.

In one or more embodiments of the present disclosure, an emote represents a single touch or click on an icon that universally relates to an identifiable emotion or feeling (i.e., an emotive) based on a user's (i.e., an Emoter's) judgment of how they feel at any given time. Moreover, in some implementations, each emote has contextual metadata associated therewith.

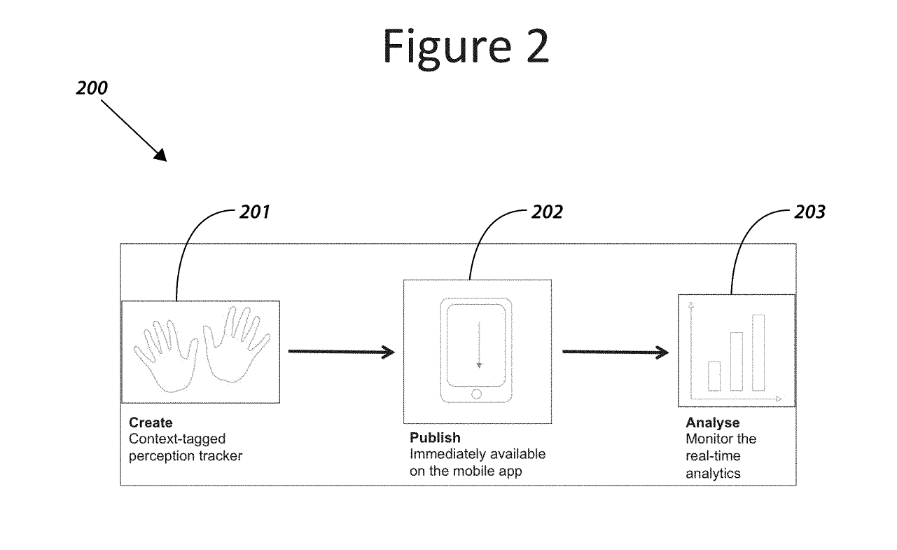

FIG. 2 is a schematic layout 200 which illustrates an overview of a solution platform's server-side process. In particular, FIG. 2 illustrates the manner in which a generic software-based perception recorder can be customized a 3-step process. As such, the present disclosure

The 3-step process may begin when a publisher creates a context-tagged perception tracker (201). Creating a context-tagged perception tracker (201) may comprise providing a manner to rapidly create a situation-specific perception recorder to suit an entity's context and requirements. For instance, when creating a context-tagged perception tracker (201), a publisher may 1) set up an activity such as an event or campaign; 2) previewing the customized recorder; and 3) publishing the customized recorder.

For example, a movie studio may create situation-specific emotives to gauge the feelings, emotions, perceptions, or the like from an audience during a movie, television show, live broadcast, or other broadcast.

After the context-tagged perception tracker are created, they may be published (202) immediately to be available on a mobile application. For instance, subscribing users may access the emotives and use them to indicate their feelings at any given time.

Next, once subscribing users emote their feelings or perceptions, a publisher can analyze the emote data (203). As such, this stage may allow publishers to monitor emote analytics real-time.

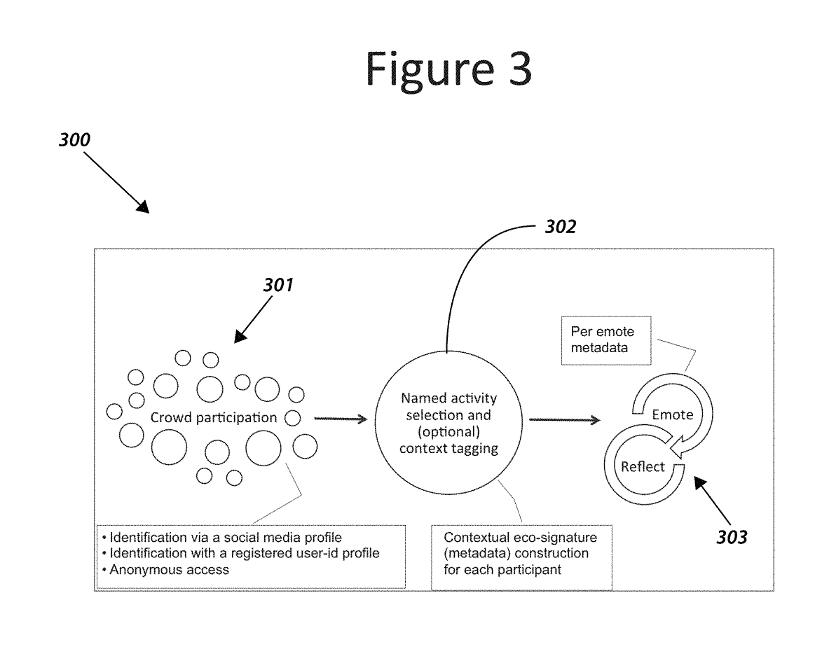

FIG. 3 is a schematic layout 300 which illustrates a solution platform's client-side process. Particularly, schematic layout 300 illustrates the manner that the software-based perception recorder can be use by a crowd of participant users (e.g., emoters) to continuously record their individual perceptions/feelings such that near real time visualization and meaningful analysis of perceptions is enabled.

Returning to the figure, the use of crowd participation (301) may be used to gauge a crowd's response to an activity or event. Subscribing users may choose to identify themselves. For example, subscribing users identify themselves via a social media profile or may identify themselves with a registered user-id profile. Alternatively, subscribing users may choose not to identify themselves and emote anonymously.

The Emoter may select the activity or event and may optionally choose to add a personalized tag/name for the selected activity. Advantageously, the present disclosure is amenable to associate a context with the Emoter, including metadata that range from demographic details, weather patterns, etc. In one or more embodiments, the contextual metadata is associated with each emote data that is sent from the client to the server-side.

In some embodiments, the participant user may be given the choice to identify themselves or to record (e.g., “Emote”) anonymously. On the client side, the Emoter is able to view and reflect upon their own emoting history and, when available, the timeline series of their emotes against the average of all emotes in a contextual scenario.

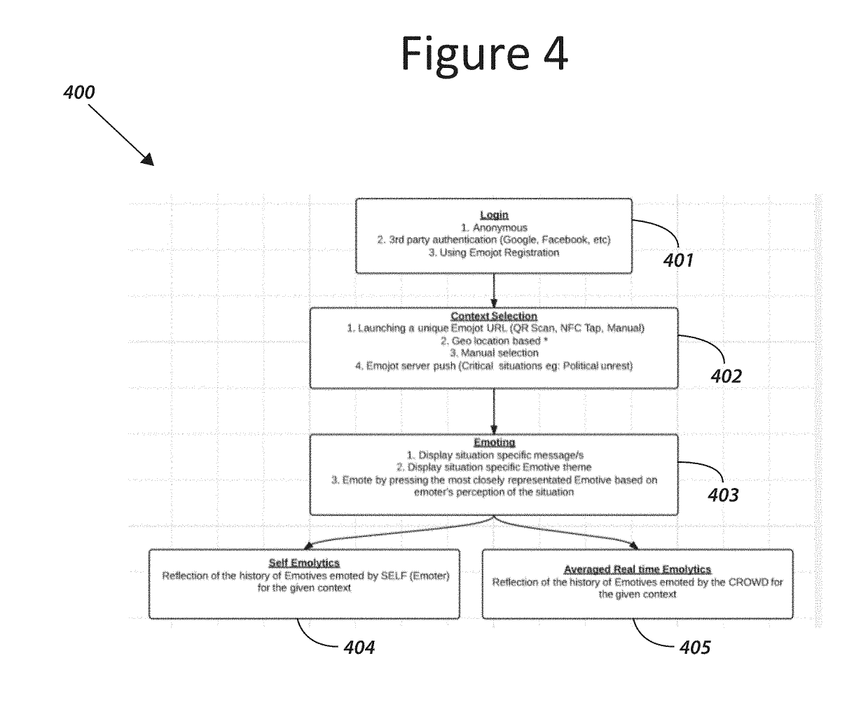

FIG. 4 is a flowchart 400 of a method of creating and publishing emotives. Flowchart 400 begins with block 401—user login. As described above, when a user logs in, they can identify themselves or do so anonymously. For example, a user may login via 3^{rd} party authentication (e.g., via a social media profile) or by using an Emojot registration.

Block 402 provides context selection by 1) launching a unique Emojot URL (e.g., QR scan, NFC Tap, Manual); 2) geo-location based; 3) manual selection; or 4) Emojot server push (e.g., critical situations such as a political unrest).

Block 403—emoting. Emoting may consist of 1) displaying situation-specific messages; 2) display situation-specific emotive themes; 3) emoting by pressing the most closely represented emotive based on emoter's perception of the situation.

Block 404—self emolytics. Reflection of the history of emotives emoted by a user (e.g., emoter) for the given context.

Block 405—average real time emolytics. Reflection of the history of emotivs emoted by the crowd for the given context.

Advantageously, this recorder may be easily customized to fit the needs of a specific perception capturing situation and instantly made available to participants as a activity-specific perception recorder via the aforementioned mechanisms. Furthermore, the present disclosure supports capturing of feelings or perceptions in an unobtrusive manner with a simple touch/selection of an icon that universally relates to an identifiable emotion/feeling (e.g., an emotive). Thus, the present disclosure is amenable to accurately capture a person's expressed feelings or perceptions regardless of language barriers or cultural and ethnic identities. Moreover, the present disclosure enables the ability to continuously capture moment-by-moment emotes via a touch interface.

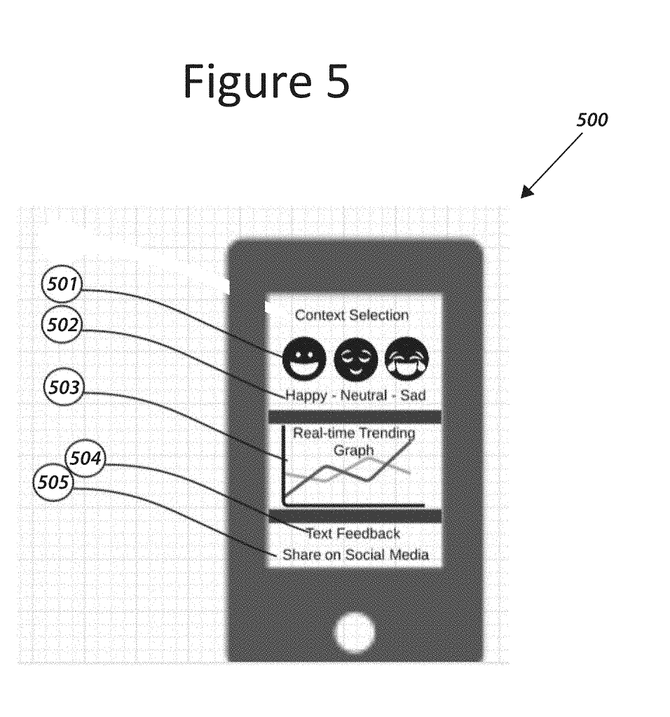

FIG. 5 is a device 500 which displays an interface 510 for selecting and publishing emotives. Interface 510 of device 500 features one or a plurality of emotives for any given context selection. In some embodiments, the context selection may represent a series of contexts/scenarios (e.g., “activities”) obtained from the server. For example, the activity may be an event, campaign, television program, movie, broadcast, or the like.

Context specific emotive themes 501 (e.g., happy, neutral, or sad) are displayed on interface 510. The present disclosure includes the ability for these emotive themes to be created and/or motivated via a server dashboard interface. For instance, an emotive theme for an opinion poll activity could have icons representing “Agree”, “Neutral”, and “Disagree,” whereas another emotive theme for a service feedback campaign activity could have icons representing “Satisfied,” “OK,” and “Disappointed.”

The description 502 of each emotive in the emotive theme are also displayed on the interface 510. The description text is a word or a few words that would provide contextual meaning for the emotive. In FIG. 5, the words “Happy,” “Neutral,”, and “Sad” appear below the three Emotives in the contextual Emotive theme.

In addition, interface 510 displays real-time “Emolytics” (e.g., Emojot analytics) as an emoter gratification method. The present disclosure permits the graph 503 to be self or crowd-averaged as a server configurable selection. When the graph 503 is set to self-averaged results, the averaged results of the emoter's emotes for the specified contextual activity will be displayed to the participant. When the graph 503 is set to crowd-averaged results, the averaged overall results of all the Emoter's emotes will be displayed to the participant.

Next, interface 510 enables text-based feedback 504. In some embodiments, the present disclosure text-based feedback 504 as a server configurable option. Similar to Twitter or Facebook, if text input is supported for a certain contextual activity, this option allows for it.

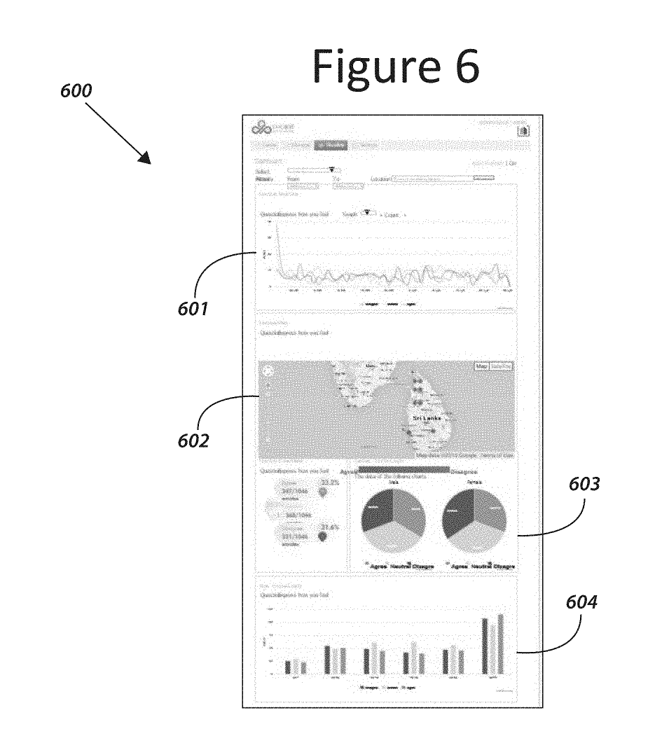

FIG. 6 is a depiction of a dashboard 600 which displays emotive analytics and related data. Dashboard 600 provides emote analytics for several context selections for a specific time period. As such, emolytics data may be generated and analyzed to determine which stimuli induces specific emotions within a subscribing user.

Within dashboard 600 is a graph 601 which displays emolytics data during a pre-specified time period. Map 602 may display emolytics data for a selected geographical region. For example, during an event or activity, the map 602 may display how emoters feel during a pre-specified time period during the activity or event.

Moreover, sections 603, 604 of dashboard 600 present additional emolytics data which illustrates how emoters were feeling during a given time period during the event or activity.

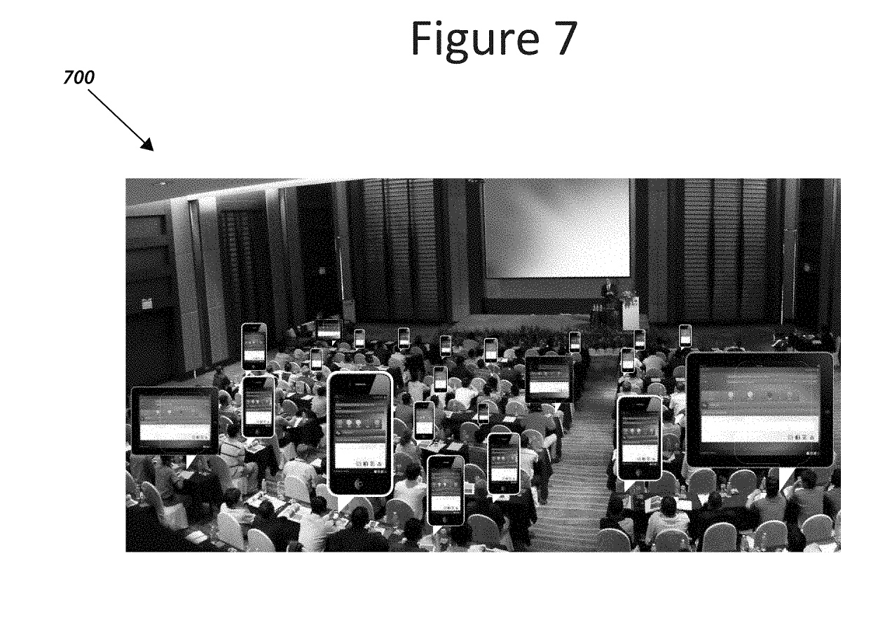

FIG. 7 is an illustration of a first use case of a perception tracker at a live event. As shown, the figure illustrates an auditorium 700 of users with a device to effect emoting. In the use case shown, users may emote how they feel during a live speaker presentation. Advantageously, during the presentation, the speaker may receive the emote data and may alternatively alter their delivery accordingly.

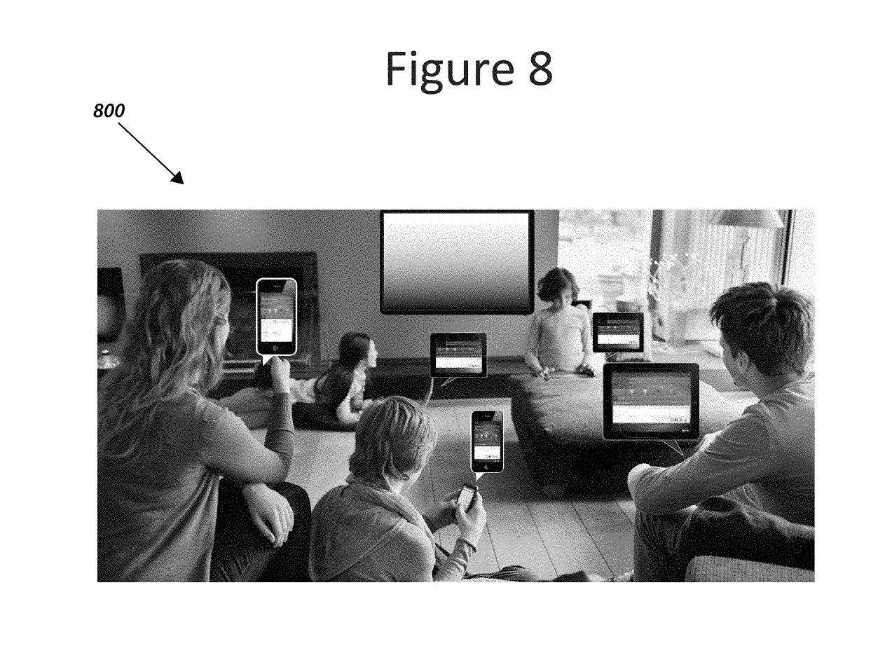

FIG. 8 is an illustration of a second use case of a perception tracker implemented for a television show. As shown, the figure illustrates a living room 800 where a family emotes via smart devices during the broadcasting of a television show. As each family member has access to a smart device, each member can emote to express their emotions, feelings, perceptions, etc. during the broadcast.

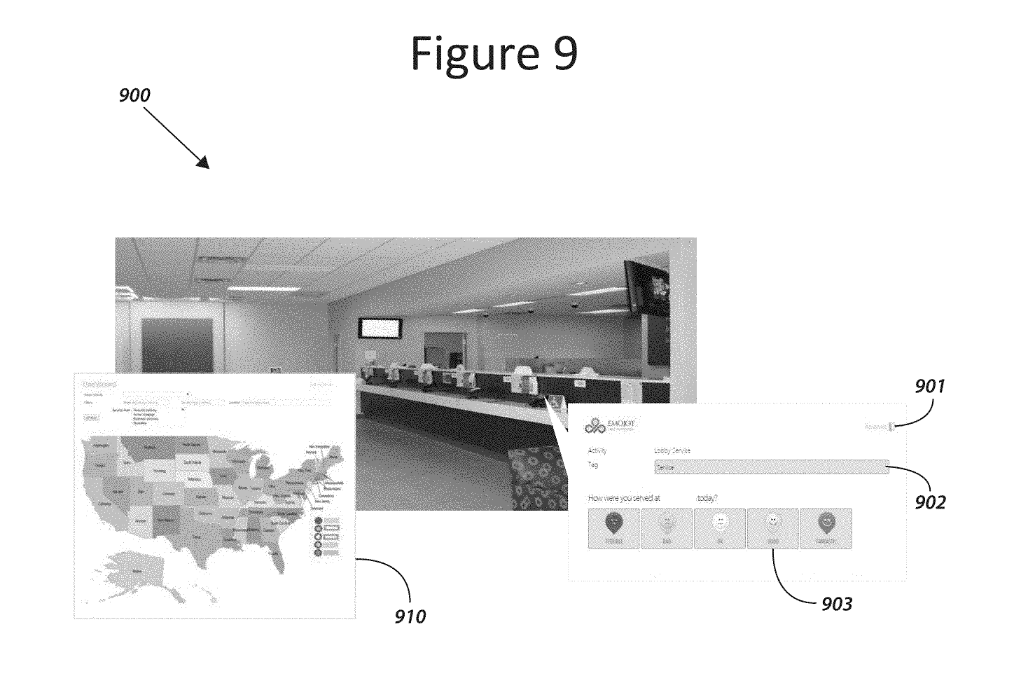

FIG. 9 is an illustration of a third use case of a perception tracker implemented for a kiosk. Advantageously, a subscribing user can emote while receiving a service. For example, FIG. 9 shows a kiosk 905 in a bank 900 where patrons can emote feelings consistent with their experience while in the bank.

Once a user logs into an application provided by kiosk 905, a user can notate their experience. For example, a user may create an emote record 901 which has various fields 902 to notate the activity, type of service, etc. Most notably, the emote record 901 provides options which indicate the customer's experience at the venue (e.g., bank). It should be understood by one having ordinary skill in the art that a user may emote their feelings via another electronic communications device.

In addition, the emote data captured by kiosk 905 may generate analytics (e.g., emolytics) which may be transmitted to bank managers, etc. For example, the emolytics data may be displayed via a dashboard 910. Dashboard 910 may display the emolytics data for several geographic regions (e.g., states). Accordingly, service providers can tailor the service offerings according to improve user feedback in needed areas.

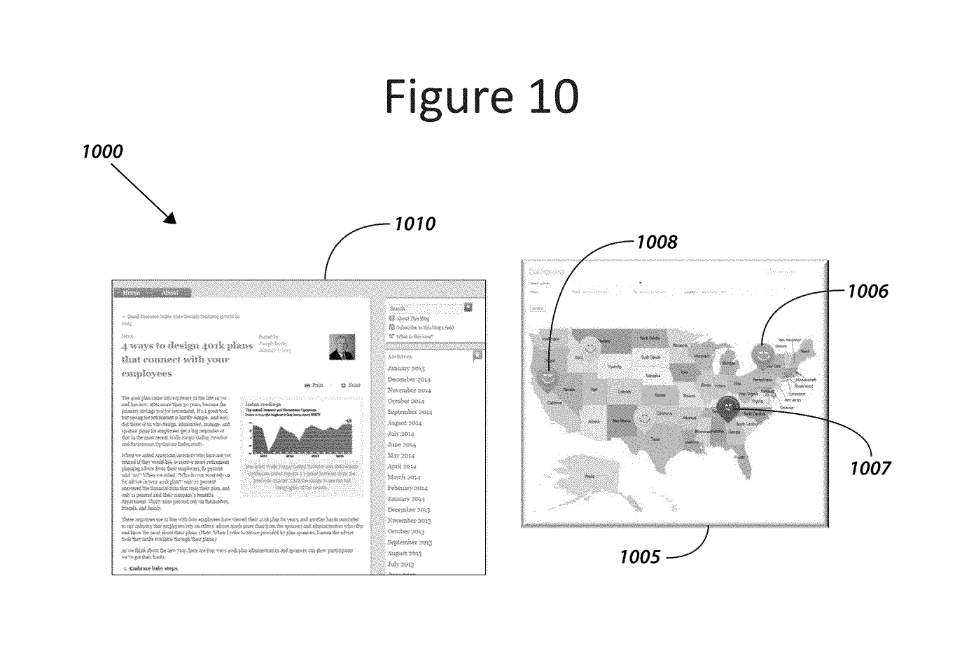

FIG. 10 is a depiction of a perception tracker embedded in a web page 1010. In particular, the perception tracker may be embedded as a widget in a web page 1010. In some implementations, the embedded widget allows the public to view the emolytics based on the administrator's settings.

Notably, display 1005 may be presented to visitors of the webpage 1010 the average emotives (or other emolytics) per geographical area (states). For example, on the map shown within display 1005, the average emotives 1006-1008 are displayed for each state.

Systems, methods, and apparatuses describing the present disclosure have been described. It will be understood that the descriptions of some embodiments of the present disclosure do not limit the various alternative, modified and equivalent embodiments which may be included within the spirit and scope of the present disclosure as defined by the appended claims. Furthermore, in the detailed description above, numerous specific details are set forth to provide an understanding of various embodiments of the present disclosure. However, some embodiments of the present disclosure may be practiced without these specific details. In other instances, well known methods, procedures, and components have not been described in detail so as not to unnecessarily obscure aspects of the present embodiments.

What is claimed is:

1. A non-transitory machine-readable storage medium containing instructions that, when executed, in response to receiving a profile of a plurality of sports teams, cause a machine to:
capture perceptions in a contextual activity.

2. The non-transitory machine-readable storage medium of claim 1 containing instructions further to provide demographic data that is associated with each perception made by a participant to be correlated during the perception

3. The non-transitory machine-readable storage medium of claim 1 containing instructions further to support offline capture of perceptions that are stored locally on a server.

4. The non-transitory machine-readable storage medium of claim 1 containing instructions further to support synchronization using wired, wireless, or physical data transmission.

5. The non-transitory machine-readable storage medium of claim 1 containing instructions further to support visual set of icons that universally relates to an identifiable emotion.

6. The non-transitory machine-readable storage medium of claim 1 containing instructions further to support haptic representation of emotives.

7. The non-transitory machine-readable storage medium of claim 1 containing instructions further to support voice-based representation of emotives.

8. The non-transitory machine-readable storage medium of claim 1 containing instructions further to support touch-based selection of a single emotive to capture a perception or feeling of an individual.
